(19) United States
(12) Patent Application Publication
(10) Pub. No.: US 2015/0038871 A1
(43) Pub. Date: Feb. 5, 2015

(54) In-Ear Proprioceptive for Relieving Temporomandibular Joint-Related Symptoms

(71) Applicant: United Sciences, LLC, Atlanta, GA (US)

(72) Inventors: Karol Constantine Hatzilias, Atlanta, GA (US); Mayoor Patel, Suwanee, GA (US); Wess Eric Sharpe, Atlanta, GA (US); William Jacob Thompson, Cornelia, GA (US)

(73) Assignee: United Sciences, LLC, Atlanta, GA (US)

(21) Appl. No.: 13/958,171

(22) Filed: Aug. 2, 2013

(51) Int. Cl. A61B 5/00; A61B 5/04; A61B 5/0488

(52) U.S. Cl. CPC A61B 5/4542; A61B 5/0488; A61B 5/486; A61B 5/7405; A61B 5/7455; A61B 5/04001
USPC 600/546; 600/590; 29/592

(57) ABSTRACT

An in-ear device with one or more proprioceptive features for providing an indication to a subject to help manage or reduce pain, discomfort, or other symptoms associated with TMJ disorder. In some embodiments, the one or more proprioceptive features are active and/or passive indicators. Also disclosed are methods of using optical scanning to create a three dimensional replication of the ear canal that is used to design a customized in-ear device.

### FIELD OF THE INVENTION

Embodiments of the invention generally relate to an in-ear proprioceptive for managing temporomandibular joint-related symptoms.

### BACKGROUND OF THE INVENTION

The temporomandibular joint (TMJ) includes a small articular disc of cartilage positioned between the mandible (lower jaw) and the temporal bone of the skull. As shown in FIGS. 2-7, the TMJ is the articulation between the two bones, allowing the lower jaw (mandible) to rotate and glide freely in various planes as the jaw opens, closes, protracts, retracts, and moves laterally and medially. The TMJ sits in front of the ear on each side of the head and abuts the ear canal (the external auditory meatus). As shown in FIGS. 2 and 7, the inferior surface of the TMJ disc 22 sits against the condyle 20 of the mandible and the superior surface of the TMJ disc sits against the fossa 24 of the temporal bone.

The TMJ moves whenever a person chews, talks, swallows, yawns, or otherwise moves his jaw and is therefore one of the most frequently moved joints in the body. As shown in FIGS. 3-5, the TMJ both rotates and translates (glides) during movement of the jaw. Specifically, the TMJ is divided into compartments: the inferior compartment, which allows the condyle 20 to rotate when the jaw first begins to open (FIG. 4), and the superior compartment, which hinges and translates (glides) with the condyle 20 as the jaw continues to open (FIG. 5).

Dysfunction of the TMJ is referred to as TMJ disorder or dysfunction (collectively, “TMD”) and can result from the TMJ becoming inflamed, injured, stressed, displaced (subluxed), dislocated, or otherwise damaged. Some people experience popping or clicking when the articular disc in the TMJ is displaced and then snaps back into position as the jaw moves; limited opening or locking of the jaw; tenderness; pain; and/or discomfort. In some cases, when a person clenches or grinds his teeth (bruxism), the condyle 20 compresses the connective tissue of the TMJ, causing inflammation of the connective tissue surrounding the TMJ (such as connective tissue 26 in FIGS. 2 and 6) and pain. In some cases, the clenching/grinding of teeth not only triggers TMJ-related discomfort, but also may contribute to the onset of TMD and to the subsequent deterioration of the joint.

It is estimated that approximately 75% of the population has at least one sign of TMD. Symptoms associated with TMD can be severe and are not always isolated to the joint itself as symptoms of TMD may present in the head, ears, neck, eyes, teeth, and/or jaw. As such, there remains a need for more effective ways to manage TMD and alleviate one or more symptoms caused from it.

### SUMMARY OF THE INVENTION

The terms “invention,” “the invention,” “this invention” and “the present invention” used in this patent are intended to refer broadly to all of the subject matter of this patent and the patent claims below. Statements containing these terms should not be understood to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to the entire specification of this patent, all drawings and each claim.

In certain embodiments, provided is a proprioceptive for reducing one or more symptoms associated with temporomandibular joint disorder. In one embodiment, the proprioceptive is configured for insertion in a subject's ear canal and may be customized based on the configuration of the subject's ear canal. In some embodiments, the proprioceptive includes an indicator that provides a cue to the subject when the subject's jaw moves past a predetermined limit of the jaw's range of motion.

According to one embodiment, disclosed is a proprioceptive for reducing one or more symptoms associated with temporomandibular joint disorder, wherein the proprioceptive is selectively configured for insertion in a subject's ear canal and includes an indicator that projects from a surface of the proprioceptive at a customized angle based on the subject's ear canal dimensions and shape and that provides a cue to the subject when the subject's jaw moves past a predetermined limit on a range of motion of the jaw.

According to another embodiment, disclosed is a custom in-ear device that substantially conforms to an ear canal of a subject having temporomandibular joint disorder when the subject's jaw is in a therapeutic position, where the therapeutic position is between an extreme open position and an extreme closed position of the jaw and wherein the therapeutic position is a position free of one or more symptoms of temporomandibular joint disorder in the subject. The in-ear device of this embodiment includes a surface having at least one feature that is customized to the subject's ear canal when the subject's jaw is in the therapeutic position and that provides a sensory indication to the subject when the subject's jaw moves past a predetermined limit in range of motion.

According to another embodiment, disclosed is a method of manufacturing an in-ear device designed to generate proprioceptive cues in the ear of a subject having temporomandibular joint disorder. In one embodiment, the method includes customizing the in-ear device so it conforms to the subject's ear canal when the subject's jaw is in a therapeutic position, where the therapeutic position is a position free of one or more symptoms of the temporomandibular joint disorder, and configuring at least one feature of the in-ear device so that the at least one feature provides a cue to the subject when the subject's jaw moves past a predetermined limit in range of motion.

According to another embodiment, disclosed is a method of reducing symptoms of temporomandibular joint disorder in a subject comprising providing, by the in-ear device, a sensory indication to the subject at predetermined intervals.

According to yet another embodiment, disclosed is a proprioceptive for insertion in a subject's ear canal, where the proprioceptive is configured to substantially deform the subject's ear canal when the subject's jaw is not in a therapeutic position, wherein the therapeutic position is between an extreme open position and an extreme closed position of the jaw and where the therapeutic position is a position free of one or more symptoms of temporomandibular joint disorder in the subject.

### BRIEF DESCRIPTION OF THE DRAWINGS

A full and enabling disclosure including the best mode of practicing the appended claims and directed to one of ordinary skill in the art is set forth more particularly in the remainder of the specification. The specification makes reference to the following appended figures, in which use of like reference numerals in different features is intended to illustrate like or analogous components.

FIG. 1 is a coronal section illustrating the anatomy of the ear.

FIG. 2 is a sagittal section illustrating the TMJ.

FIGS. 3-5 illustrate the movement of the TMJ as the jaw opens.

FIG. 6 is a transverse section showing the positioning of the TMJ relative to the ear canal.

FIG. 7 is another coronal section showing the TMJ.

FIG. 8 is a perspective view of a custom designed TMD proprioceptive according to one embodiment.

FIG. 9 is another view of the proprioceptive of FIG. 8.

FIG. 10 is another view of the proprioceptive of FIG. 8.

FIG. 11 is a perspective view of a proprioceptive according to another embodiment.

FIG. 12 is a perspective view of a proprioceptive according to another embodiment.

FIG. 13 is a perspective view of a proprioceptive according to another embodiment.

FIG. 14 is a lateral sagittal view of the proprioceptive of FIG. 13.

FIG. 15 is a lateral sagittal view of a proprioceptive according to another embodiment.

FIG. 16 is a sagittal section showing the distribution of the trigeminal nerve.

### DETAILED DESCRIPTION OF THE DRAWINGS

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

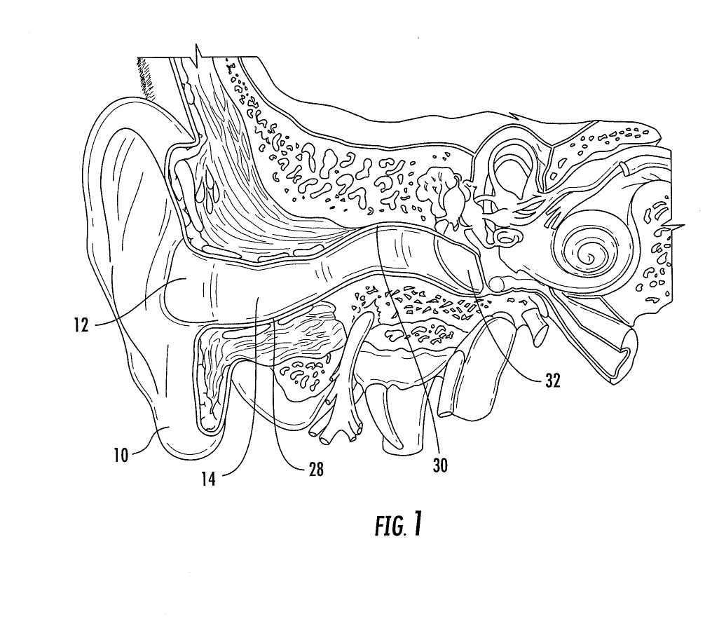

As shown in FIG. 1, the ear canal (auditory canal) 14 extends from the concha 12 and forms a generally-S shaped curve that has constrictions, one at a first bend 28 and another at a second bend 30. These bends help prevent foreign objects from reaching and damaging the ear drum (tympanic membrane) 32. FIG. 1 illustrates the ear canal generally, but much like fingerprints, each person's ear canal is unique.

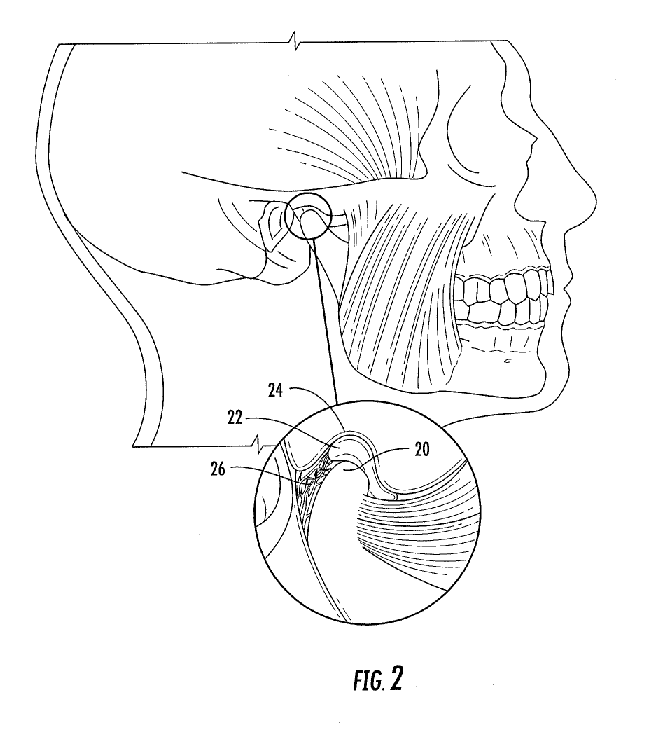

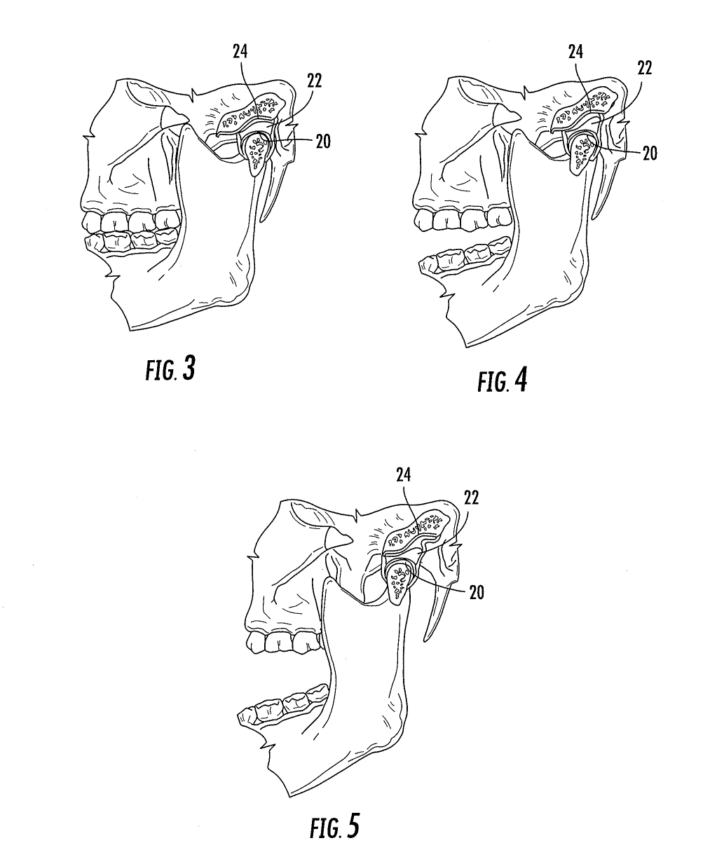

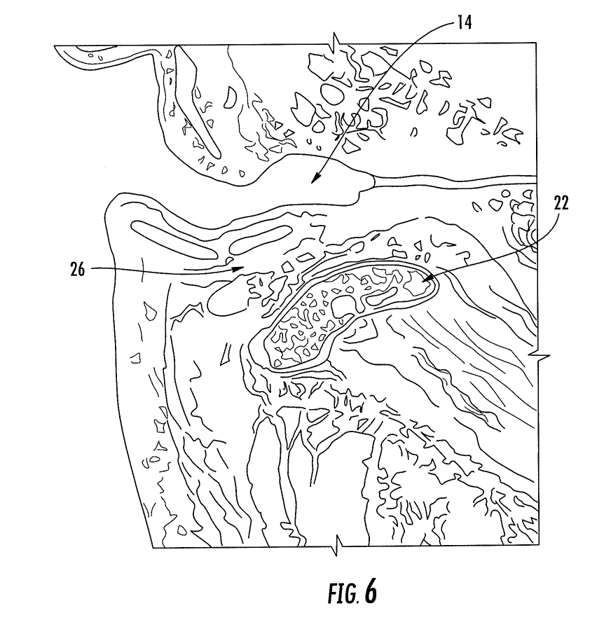

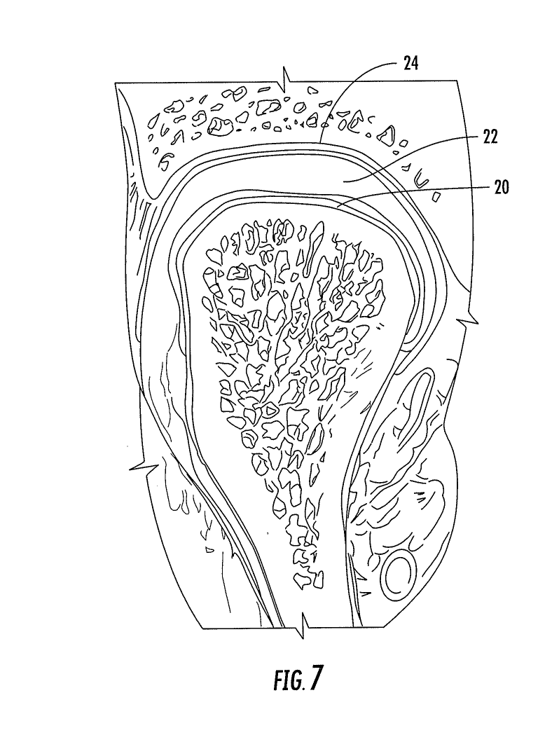

The TMJ is positioned in front of the ear canal, as illustrated in FIGS. 2 and 6. Disclosed herein are various applications that capitalize on the TMJ's proximity to the ear canal to influence the operation of the TMJ and to help alleviate or prevent symptoms and discomfort associated with TMD.

In-Ear Proprioceptive

Disclosed is an in-ear device (a proprioceptive) having one or more proprioceptive features for alleviating or reducing one or more TMJ-related symptoms in a subject. As used herein, proprioception refers to a conscious or subconscious indication to a subject that influences the subject's perception and, in some cases, the subject's behavior. In some instances, proprioception influences a subject's behavior even if the subject is not consciously aware of it. In particular, in some embodiments, the in-ear device includes one or more features that influence the subject's perception. In some embodiments, the one or more features provide one or more proprioceptive cues or indicators to the subject informing the subject to alter his movements to avoid or reduce pain associated with the TMJ and/or to avoid or reduce deterioration of the TMJ. As described in more detail below, these indicators can be passive or active or any suitable combination of both.

In-Ear Proprioceptive with Active Indicators

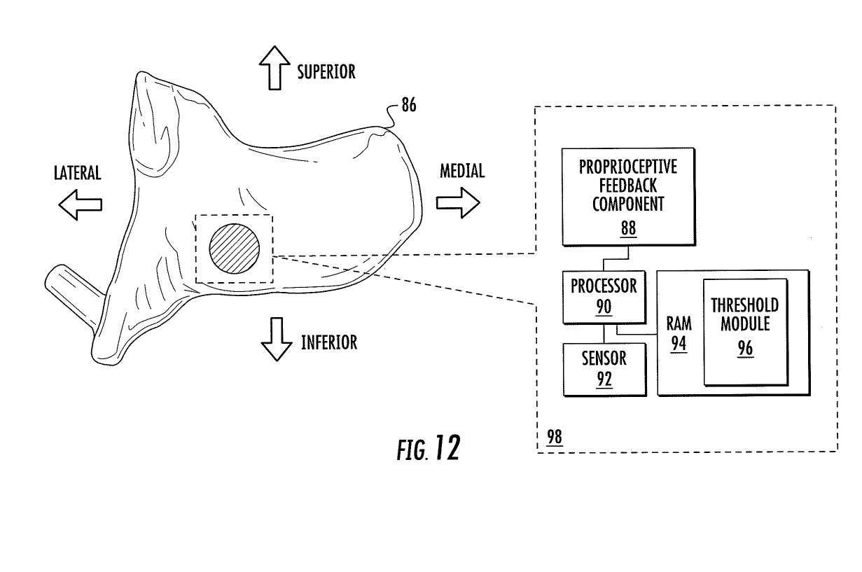

In some embodiments, the one or more proprioceptive features are mechanical and/or electrical sensors. These sensors may be referred to as active indicators. One non-limiting embodiment of an in-ear device 90 with at least one sensor component 92 is shown in FIG. 12. As shown, the sensor component 92 may include a proprioceptive feedback component 88, a processor 90, one or more sensors 92, random access memory (RAM) 94, and a threshold module 96. Sensor 92 may include any number of sensors and may be any suitable sensor, such as a force sensor, an accelerometer, a voltage sensor, and/or any other suitable sensor. In one non-limiting embodiment, the one or more sensors 92 measures a physical quantity and sends information associated with the physical quantity to processor 90. The processor 90 uses information stored in the threshold module 96 to determine if the physical quantity exceeds a predetermined threshold stored in memory. If the physical quantity exceeds the predetermined threshold, the processor 90 instructs the proprioceptive feedback component 88 to generate a suitable signal. In this embodiment, the actor in this active configuration is a software module that compares the value received from the sensor to the threshold held in memory and determines whether to send a proprioceptive signal.

In some embodiments, proprioceptive feedback component 88 is a vibration motor or speaker or any other component capable of generating a suitable signal or earcon to the subject as discussed below. In some embodiments, the sensor component 92 is an analog system that does not require a processor or memory.

In one embodiment, sensor 92 may be a force sensor that is configured to measure the force exerted by the jaw to determine when the jaw is being clenched and/or the teeth are grinding or the jaw has otherwise moved too far and the subject is approaching the point of TMJ-related symptoms (e.g., pain or discomfort). Specifically, when the jaw is clenched and/or the teeth are grinding, the shape and/or position of the subject's ear canal changes, inherently exerting a force on the in-ear device. The force sensor can be used to measure the force exerted on the in-ear device when the jaw is clenched and/or the teeth are being ground. The in-ear device then can be programmed so that, when the force sensor detects force approaching this predetermined measurement in use, a transducer transmits an appropriate signal to the subject.

The signal generated by the proprioceptive feedback component 88 may be a vibration, an audio signal, or any other suitable signal that indicates to a subject that he is clenching/grinding his teeth and that he is approaching the point of invoking TMJ-induced symptoms and/or deterioration. In some embodiments, the signal is generated when the subject's jaw is clenched or he is grinding his teeth, or when he closes his jaw past a predetermined threshold/limit of movement. In some embodiments, the predetermined limit of movement corresponds to the subject's jaw position associated with one or more symptoms of TMD.

In some cases, the force sensor alone may be incapable of detecting movement of the jaw past the predetermined threshold and therefore may be insufficient to provide the desired feedback to the subject. In these situations, the sensor 92 may include an accelerometer (instead of or in addition to) the force sensor that monitors the rate of motion of the jaw. When the acceleration of the jaw exceeds a certain threshold (such as when the jaw is clenched and/or opened too wide or otherwise moved to an extreme point with sufficient acceleration), the accelerometer can send a signal to the subject indicating that the rate of change in the jaw position needs to be changed to avoid or reduce one or more TMJ-related symptoms. The accelerometer also may be configured to detect joint sounds and provide feedback based on the detected joint sounds.

As shown in FIG. 16, the mandibular branch (V₃ branch) 34 of the trigeminal nerve runs near the TMJ. The mandibular innervates the muscles involved in mastication (chewing). During clenching and grinding, which as described above are factors that cause TMJ dysfunction, these muscles are activated through efferent electrical signals through the mandibular nerve. In one embodiment, the sensor 92 may be a voltage detector that measures the voltage across the mandibular nerve from inside the ear canal. When the voltage reaches a predetermined threshold, the device transmits a signal (such as an auditory signal or a vibration or other suitable signal) to the subject. The voltage detector may also be used to measure the voltage across a muscle (such as the masseter, temporalis, or pyerygoid muscles) to determine whether the subject is clenching or grinding.

In some embodiments, the in-ear device does not include an input signal, but is configured to emit a signal that is time dependent. For example, the in-ear device can be configured to send a signal to the subject at predetermined intervals. For example, a vibration, audio, or other suitable signal emitted at predetermined temporal intervals may provide a subject with feedback to consciously assess and correct the positioning of his jaw to relieve stress on the TMJ and reduce inflammation and deterioration.

In some embodiments, these active proprioceptive mechanical and/or electronic indicators replace one or more passive proprioceptive features described below. In other embodiments, these mechanical and/or electronic signals are used in addition to the one or more passive proprioceptive indicators described below. In each case, the features are selected to meet the particular needs of the subject.

In-Ear Proprioceptive with One or More Passive Indicators

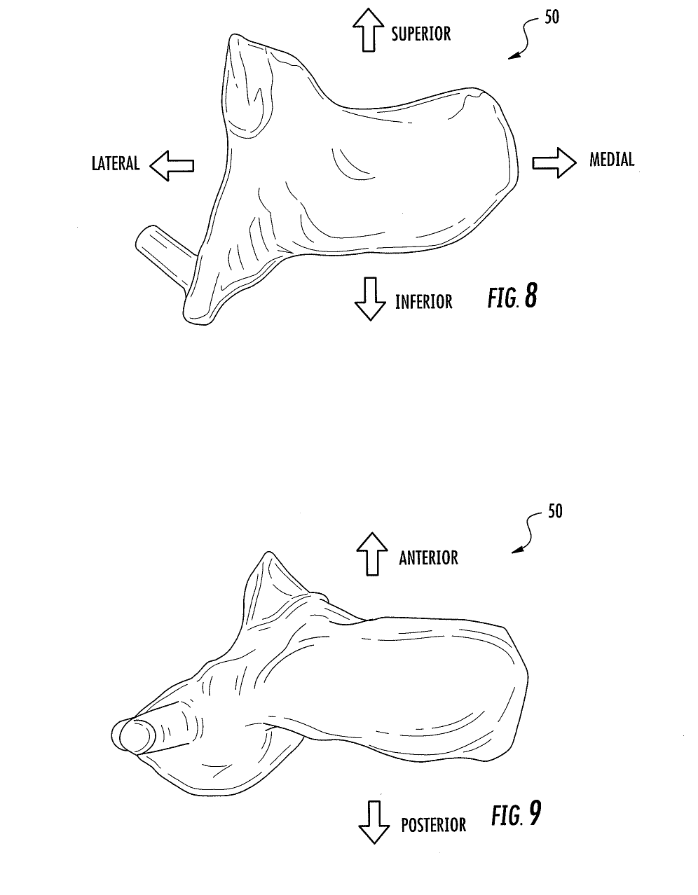

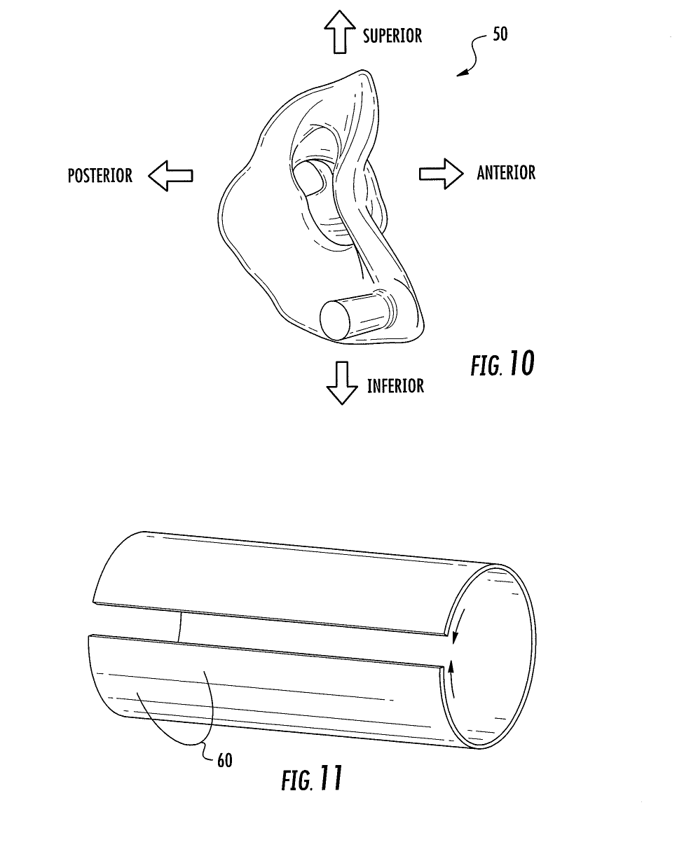

In some embodiments, the in-ear device is custom-designed so that it substantially conforms to a particular subject's ear canal when the jaw is in a particular location and/or so that it deforms the subject's ear canal when the jaw moves in a predetermined way. A non-limiting example of a custom-designed in-ear device is shown in FIGS. 8-10 as in-ear device 50.

Generally, the cross-sectional area and configuration of the ear canal changes as a subject opens and closes or otherwise moves his jaw. In addition, the ear canal may translate in any direction as the subject moves his jaw. With some people, the cross-sectional area of the ear canal decreases as the jaw moves from its therapeutic or optimal position to the closed position and/or as the jaw moves from its therapeutic position or optimal to the open position. Moreover, with some subjects, the subject's jaw moves in an anterior-posterior and/or superior/inferior direction as the subject's jaw moves from its therapeutic or optimal position.

The therapeutic or optimal position of the jaw is one that changes a subject's symptomatic and/or dysfunctional maxillomandibular relationship to one that is more normal, less symptomatic and/or more fully functional, and in some cases involves repositioning the mandible vertically, anteroposteriorly and/or transversely to the extent necessary. The therapeutic or optimal position of the jaw varies from subject to subject, but can be determined using any suitable, conventional method, some examples of which are given below. In some cases, the therapeutic or optimal position is a neutral, more asymptomatic position of the jaw that helps relieve stress on the TMJ disc and surrounding tissues. In some cases, the therapeutic position is between an extreme closed position and an extreme open position of the jaw and is a position that reduces one or more symptoms of the temporomandibular joint disorder. It is within the skill of one of skill in the art to select the therapeutic or optimal jaw position for any given subject.

For some subjects, an in-ear device situated within the ear canal will mechanically exert forces on the ear canal when the cross-sectional area of the ear canal decreases and/or when the ear canal translates, providing proprioceptive cues. When the cross-sectional area of the ear canal decreases beyond a predetermined value, the forces exerted on the ear canal as the in-ear device deforms the ear canal may be sufficient to provide an indication (such as a sensation of discomfort or fullness in the ear canal) to the subject that he has closed (or opened) his mouth or otherwise moved his jaw to the selected TMJ threshold, and that he should stop movement to avoid or reduce one or more TMJ-related symptoms and/or inflicting further damage on the TMJ.

In some embodiments, the in-ear device is configured and/or dimensioned so that the forces exerted on the ear canal are sufficient to provide the subject with the sensory indication when the subject begins clenching/grinding his teeth and/or when he closes his jaw beyond a predetermined threshold. In this way, the device itself is configured to have a proprioceptive feature that functions to provide mechanical resistance and alert a subject to alter the movement of his jaw to prevent or reduce TMJ-related symptoms and/or deterioration. This proprioceptive feature is sometimes referred to as a passive indicator.

In some cases, continuous pressure or regular proprioception causes the subject's muscles to relax (either through proprioception or through pressure caused by deformation of the ear canal). Moreover, in some cases, deforming the subject's ear canal or otherwise using an in-ear device to exert pressure on the ear canal may help relieve pain associated with TMD. According to a theory known as the Gate Theory, activating diameter nerve fibers by grabbing, holding, applying pressure to, and/or rubbing a painful site can inhibit (suppress) pain sensation at the spinal cord level from that segment of the body. As such, the in-ear devices described herein can be used to apply pressure in a way that reduces pain or other symptoms associated with TMD.

The in-ear device may be used in one or both ears depending on the needs of the subject. In some embodiments, the in-ear device is customized to conform to a particular subject's unique ear canal, as discussed below.

The in-ear device may be formed of any suitable material, such as, but not limited to, polymers such as polypropylene (PP), polyethylene (PE), polytetrafluoroethylene (PTFE), acrylic, acrylonitrile budadiene styrene (ABS), polyether ether ketone (PEEK), silicone, thermoplastic elastomers such as polyurethane, or any other suitable material. In some cases, the material is selected so the in-ear device is capable of being compressed for insertion into the ear and so that the in-ear device expands to its original state after a predetermined period of time. In some embodiments, the in-ear device is formed of a heat-dependent shape memory polymer or alloy. One non-limiting example is a nickel titanium alloy (nitinol).

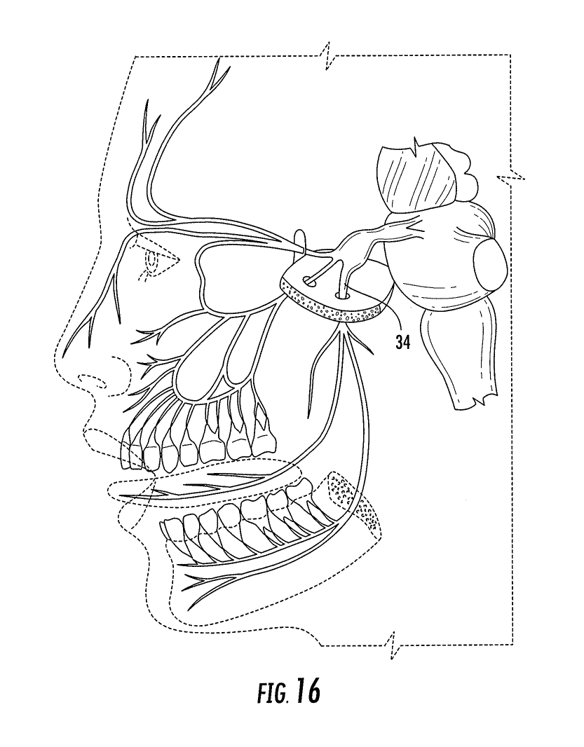

As mentioned, the in-ear device may be formed of any suitable material, including, for example, a combination of rigid and soft materials, as shown in FIG. 15. The in-ear device may have any suitable durometer, for example, a durometer between approximately 20A-80A. The durometer of the device can be customized based on the particular hardness and elasticity of the subject's ear canal. In some embodiments, subsurface imaging or any other suitable technique may be used to determine the hardness and elasticity of the subject's ear canal. In the non-limiting embodiment illustrated in FIG. 15, the in-ear device is formed of a combination of rigid and soft materials. For example, the inner material 82 may be a rigid or semi-rigid material (e.g., but not limited to, a material having a durometer of approximately 60A-80D) that provides support to the in-ear device, while the outer material 84 may be a relatively soft and flexible material (e.g., but not limited to, a material having a durometer of approximately 1000-40A) that is relatively comfortable when in contact with the subject's ear canal. In some embodiments, the in-ear device has a hollow center 80, as shown in FIG. 15.

The combination of materials may also be selected so that the in-ear device selectively expands. In particular, the materials may be selected so that the device only expands in portions that correspond to areas of the ear canal where deformation is desired (i.e., where it is desired that the forces supplying the sensory indication be supplied). The rigidity of the material can also be selected to limit TMJ motion, as an increase in rigidity limits more motion than a less rigid or relatively soft material.

In some embodiments, as shown in FIG. 11, the in-ear device may be generally C-shaped or have a generally C-shaped internal cavity or sound channel. A generally C-shaped device as shown in FIG. 11 may facilitate compression of the in-ear device before insertion and therefore facilitate the insertion of the device into the ear canal. The split C-shaped nature of the device illustrated in FIG. 11 may also be configured to help direct the forces associated with the sensory indication to the subject. In some cases, the split nature of the device provides a spring-like effect that helps orient the device properly within the ear canal. The generally C-shaped device is optionally customized to conform to the subject's ear canal, thus providing a device that is customized, but is easily insertable. As one non-limiting example, the flexural modulus of the generally C-shaped device may be selected to vary how much force the device applies to the ear canal.

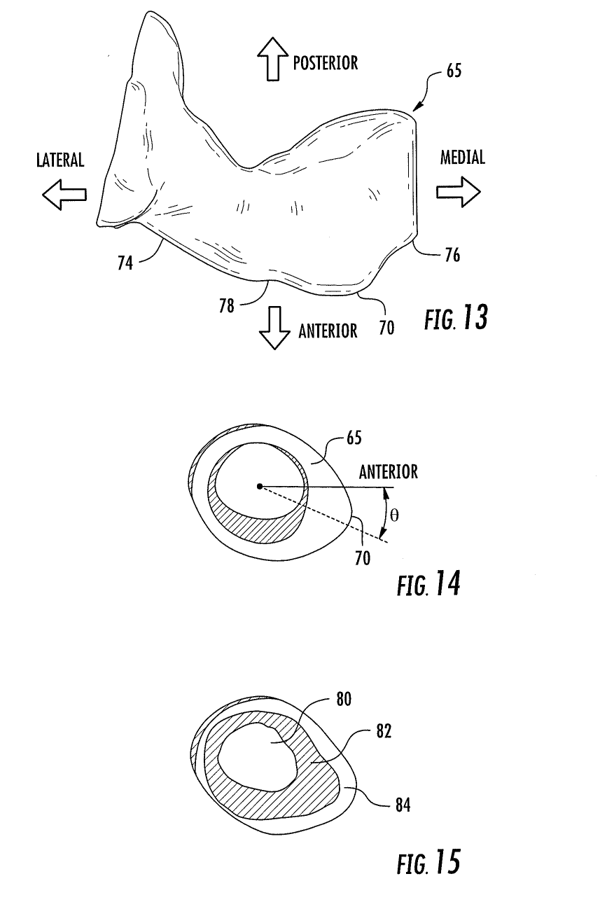

In some embodiments, the in-ear device includes a protrusion 60 that protrudes from the device, an embodiment of which is shown in FIG. 11, or a protrusion 70 as shown in FIG. 13. Generally, the protrusion may be positioned along the in-ear device at a customizable relative distance. For example, the protrusion may be positioned along the in-ear device such that it is situated within either the first bend 16 or the second bend 18 of the ear canal 12 when the device is inserted in the ear canal 14. As shown in FIG. 13, protrusion 70 may be positioned along in-ear device 65 a predetermined distance from any suitable landmark such as the first bend 78, the second bend 76, or the aperture 74.

In some cases, the protrusion 60 is configured to project from the in-ear device at a predetermined angle that corresponds to the configuration of the particular subject's ear canal. In this way, along with the location of the protrusion along the in-ear device, the angle θ (see FIG. 14) from which the protrusion projects from the in-ear device may be customized based on the particular subject's ear canal.

In some embodiments, more than one protrusion is included. In some cases, the first protrusion is positioned along the device such that it is situated within the first bend of the ear canal when the in-ear device is inserted in the ear canal and the second protrusion is positioned along the device such that it is situated within the second bend of the ear canal when the device is inserted in the ear canal.

Alternatively, the one or more protrusions may be positioned at any other suitable location along the in-ear device depending on the configuration of the particular subject's ear canal. For example, the protrusion 60 may be positioned along the in-ear device so that it is situated within the portion of the particular subject's ear canal that expands/contracts the most throughout the jaw movement (i.e., the segment of the canal with the most mobility). Because in these embodiments the protrusion 60 is situated within the portion of the ear canal with the most expansion/mobility, a sensory indication is provided to the subject based on the forces exerted by the protrusion 60 when the subject begins to clench/grind his teeth or has otherwise reached his jaw's threshold for opening and/or closing or other movement. In some cases, the protrusion is also referred to as a passive indicator, as it is the interaction of the in-ear device itself with the ear canal that provides the sensory indication.

Optionally, the protrusion includes a durometer, which may be selected so that it has a rigidity sufficient to exert force on the ear canal when the subject is grinding/clenching his teeth and/or his jaw is opened too wide or otherwise moved too far and so as to provide a sensory indication to the subject to alter the movement of his jaw to avoid or reduce one or more TMJ-related symptoms. The durometer of the protrusion may be customized based on the configuration of the particular subject's ear canal and the sensitivity of his sensory receptors. In some non-limiting embodiments, the durometer of the protrusion is between approximately 60A-80D.

In some cases, the protrusion is added if the forces exerted by the in-ear device are insufficient to provide the particular subject with a sensory indication that he should limit his jaw's movement or if more precise control is needed or desired. Depending on the needs of the subject, the in-ear device can include any suitable number and type of passive and/or active indicators. In some embodiments, the in-ear device does not include any passive or active indicators, but is customized based on the particular subject's ear canal to deform the subject's ear canal in a way that alleviates one or more symptoms of TMD.

Method of Designing a Custom In-Ear Device

As shown in FIGS. 8-10, the in-ear device may be one that is customized based on the particular subject's ear canal. In this way, the in-ear device conforms to at least a portion of the particular subject's ear canal. The customized device can be designed using any suitable method, such as, but not limited to, scanning the ear canal to create a 3D replication of the ear canal. In some cases, the in-ear device may also be customized based on scans of the outside of the jaw. For example, U.S. Ser. No. 13/417,767, filed Mar. 12, 2012 and titled “Optical Scanning Device”; Ser. No. 13/417,649, filed Mar. 12, 2012 and titled “Otoscanning with 3D Modeling”; Ser. No. 13/586,471, filed Aug. 15, 2012 and titled “Video Otoscanner with Line-of-Sight of Probe and Screen”; Ser. No. 13/586,411, filed Aug. 15, 2012 and titled “Otoscanner with Fan and Ring Laser”; Ser. No. 13/586,459, filed Aug. 15, 2012 and titled “Otoscanner with Camera for Video and Scanning”; Ser. No. 13/586,448, filed Aug. 15, 2012 and titled “Otoscanner with Pressure Sensor with Compliance Measurement”; and Ser. No. 13/586,474, filed Aug. 15, 2012 and titled “Otoscanner with Safety Warning System,” the contents of all of which are incorporated herein by reference in their entireties, disclose suitable methods of scanning the ear canal, building a three-dimensional shape, and designing a customized in-ear device based on the generated three-dimensional shape. In-ear device 50 shown in FIGS. 8-10 is a non-limiting example of a customized in-ear device designed from a 3D shape created from optical scanning of the ear canal.

According to some embodiments, the in-ear device can be customized based on the shape of the subject's ear canal when the subject's jaw is in the therapeutic or optimal position. The therapeutic or optimal position of the jaw can be determined using any desired conventional method. For example, some believe that the therapeutic or optimal position of the jaw is when the jaw is in a forward position. One skilled in the art will appreciate that the therapeutic or optimal jaw position may be determined using any one of a number of known methods. For example, the therapeutic or optimal position may be determined by indexing of the jaw. This position may also be determined by aligning the lower jaw and the upper jaw in a predetermined manner such as at their midpoints. The position may alternatively be determined using the swallow technique, selecting the position phonetically (when the jaw is positioned as certain sounds are made), or by arbitrarily selecting what appears to be the therapeutic or optimal position based on visual inspection. Once the jaw is in this therapeutic or optimal position, the position can be indexed with wax or bite registration material. This wax or bite registration can be used to maintain the jaw in its therapeutic or optimal position. While the jaw is maintained in this therapeutic or optimal position, the ear canal may be scanned as described above and a 3D image of the ear canal when the jaw is in the therapeutic or optimal position may be generated. In this way, the device is custom designed so that it conforms to the ear canal when the jaw is in the therapeutic or optimal position and so that it deforms the ear canal when the jaw moves out of the therapeutic or optimal position and/or moves past a predetermined threshold. As discussed above, the rigidity or softness of the device can be varied to meet the particular needs of the subject.

In some embodiments, the in-ear device may be customized using scans of the outside of a subject's jaw, either alone or in combination with scans of the subject's ear canal. In addition to scanning, parameters may also optionally be used to customize the in-ear device to the particular subject. For example, the subject's facial type, height, gender, age, demographics, weight, occupation, and other demographic information can be used to help customize the in-ear device. In some cases, the subject's stage in what is known as the Piper classification system for TMD or other parameters or TMD are used to customize the in-ear device. Etiological or pathophysiological parameters or other information from the study of information sciences may be used to customize the in-ear device. Any or all of these various parameters, along with feedback provided by the subject, may be used in a feedback loop to further customize the in-ear device.

In embodiments where the device is customized to the particular subject's ear canal based on the configuration of the ear canal when the subject's jaw is in the therapeutic or optimal position, the device will substantially conform to the subject's ear canal when the subject's jaw is in the therapeutic or optimal position. In this way, the subject will not receive any sensory indications associated with the in-ear device when the subject's jaw is in the therapeutic or optimal position. When the jaw goes beyond the therapeutic or optimal position by a certain predetermined amount (for example, when the subject begins to clench/grind his teeth or closes his jaw beyond the therapeutic or optimal position), the device provides a sensory indication to the subject as described above. In particular, in cases where the subject's ear canal decreases in cross-sectional area when the jaw is closed, the in-ear device will no longer substantially conform to the ear canal when the jaw is closed, causing the in-ear device to exert force on the ear canal when the jaw is clenched or the teeth are grinding (and in some embodiments, to substantially deform the subject's ear canal) and provide a sensory indication to the subject that he should alter movement or position of his jaw to avoid or reduce TMJ-related symptoms.

Also disclosed is a method of scanning the jaw in its therapeutic or optimal position, its closed position, its open position, or any combination thereof to track how the dimensions of that particular subject's ear canal changes. These scans can then be used to determine the positioning of one or more protrusions as described above, including the location of that particular subject's first and second bends. Moreover, if the scans indicate that the cross-sectional area of the subject's ear canal decreases when the jaw is closed and/or open, it might be determined that passive detection as described above is sufficient. On the other hand, if the scans indicate that the cross-sectional area of the subject's ear canal does not decrease when the jaw is closed and/or open, it might be determined that active detection in form of accelerometer, voltage sensor, or other suitable sensor should be incorporated into the in-ear device. Essentially, 3D scanning of the ear can be used to determine the appropriate in-ear device solution for the subject, including the dimensions and/or overall shape of the device and whether to include active indicators in addition to passive indicators.

As described above, tissue hardness and elasticity, ear canal translation, ear canal cross-sectional area change, and subject-specific pain threshold are all input specifications that can be used to create a custom-designed in-ear device for the treatment of TMD from the ear canal. In some cases, 3D scans coupled with post-processing allow for relative position and volume analysis. In addition, mechanical factors also can be analyzed to create a custom in-ear device. For example, output parameters such as protrusion radius, relative position, angle, durometer, and wall thickness depend on movements of the mandibular condyle and can affect canal dynamics. As such, 3D scans may not able to completely detect movement of the mandibular condyle since tissue hardness and elasticity attenuates visual motion inside the canal. Moreover, pressure needed for proprioceptive feedback differs from subject to subject, along with tissue hardness and elasticity and ear canal dynamics, and a device that creates unnecessary pain should be avoided. Because sensation and pain are subjective, these factors can be considered individually during the creation of a custom in-ear device. To help account for these various factors, a measurement device may be used in conjunction with the methods described above to help design a custom in-ear device. In one embodiment, the device includes a distal end that extends bilaterally and includes an indicator that measures the depth from the ear canal aperture, diameter of the ear canal, and/or angle of application. In some embodiments, the device includes a tension adjuster to determine hardness and elasticity of the tissue, which may help determine the optimum parameters of sensation or pain needed for the in-ear device. In some embodiments, the measurement tool may include electrical and computing components such as force sensors, orientation sensors, and interface devices.

It should also be understood that the subject matter described herein may be incorporated into any suitable in-ear device such as hearing aids, ear buds, hearing protection devices, and so forth.

Method of Treating or Preventing One or More TMJ-Related Symptoms

Disclosed is a method of treating TMD in a subject by providing the described device to the ear canal of the subject. Optionally, the device is provided to the ear canal during the day when the subject is awake and a mouth guard is provided at night when the subject is asleep and not as receptive to the signals provided by the one or more proprioceptive features.

Also disclosed is a method of treating one or more symptoms of TMD in a subject by creating a customized in-ear device as described above to influence the positioning of the jaw. In particular, the in-ear device can be used to help keep the upper and lower teeth separated so the jaw can move without occlusal (dental) interferences. Over time, the custom in-ear device can be replaced with a new in-ear device that is customized based on the adjusted position and/or movement of the jaw. Over time, the iterative in-ear devices can help influence the movement of the jaw back into its therapeutic or optimal position by accommodating changes in the jaw's position. Although the TMJ disc itself might not reposition into its original location, the use of the in-ear devices can be used to encourage remodeling or even pseudodisc formation to prevent or reduce TMJ-related pain.

Kits

Further provided is a method of treating TMD in a subject wherein the customized in-ear device is modified over time to provide a series of devices, where each device in the series is customized to the subject.

Specifically a kit comprising multiple pairs of in-ear devices may be selectively configured for insertion in the subject's ear canal, where each pair of the in-ear devices is designed to provide progressive adjustment of the temporomandibular joint disorder of the subject.

The foregoing is provided for purposes of illustrating, explaining, and describing embodiments of the present invention. Further modifications and adaptations to these embodiments will be apparent to those skilled in the art and may be made without departing from the scope or spirit of the invention. Different arrangements of the components depicted in the drawings or described above, as well as components and steps not shown or described are possible. Similarly, some features and subcombinations are useful and may be employed without reference to other features and subcombinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

We claim:

1. A proprioceptive for reducing one or more symptoms associated with temporomandibular joint disorder, wherein the proprioceptive is selectively configured for insertion in a subject's ear canal and comprises an indicator that projects from a surface of the proprioceptive at a customized angle based on the subject's ear canal dimensions and shape, wherein the indicator provides a cue to the subject when the subject's jaw moves past a predetermined limit on a range of motion of the jaw.

2. The proprioceptive of claim 1, wherein the proprioceptive is configured to substantially conform to the subject's ear canal when the subject's jaw is in a therapeutic position, wherein the therapeutic position is between an extreme closed position and an extreme open position of the jaw and wherein the therapeutic position is a position that reduces the one or more symptoms of the temporomandibular joint disorder.

3. The proprioceptive of claim 2, wherein the proprioceptive is configured to substantially deform the subject's ear canal when the subject's jaw is not in the therapeutic position.

4. The proprioceptive of claim 1, wherein the cue is generated by forces exerted by the indicator on the ear canal and wherein the cue is a physical sensation.

5. The proprioceptive of claim 1, wherein the indicator is compressible.

6. The proprioceptive of claim 1, wherein the indicator comprises a protrusion.

7. The proprioceptive of claim 6, wherein the protrusion is configured to align with either a first bend or a second bend of the subject's ear canal.

8. The proprioceptive of claim 6, wherein the protrusion is configured based on demographics of the subject.

9. The proprioceptive of claim 6, wherein the protrusion is configured based on a three-dimensional optical scan of the subject's ear canal.

10. The proprioceptive of claim 1, wherein the proprioceptive is configured to conform to the subject's ear canal with a three-dimensional optical scan of the subject's ear canal.

11. The proprioceptive of claim 1, wherein the indicator comprises a durometer and a flexural modulus that are selected based on particulars of the subject's ear canal.

12. The proprioceptive of claim 1, wherein the proprioceptive includes a generally C-shaped interior cavity.

13. A custom in-ear device that substantially conforms to an ear canal of a subject having temporomandibular joint disorder when the subject's jaw is in a therapeutic position, wherein the therapeutic position is a position free of one or more symptoms of temporomandibular joint disorder in the subject, wherein the in-ear device comprises:
a surface comprising at least one feature that is customized to the subject's ear canal when the subject's jaw is in the therapeutic position, wherein the at least one feature provides a sensory indication to the subject when the subject's jaw moves past a predetermined limit in range of motion.

14. The custom in-ear device of claim 13, wherein the at least one feature comprises a protrusion that projects from the surface of the device at a customized angle.

15. The custom in-ear device of claim 14, wherein the protrusion is configured to align with either a first bend or a second bend of the subject's ear canal.

16. The custom in-ear device of claim 13, wherein the at least one feature is compressible.

17. The custom in-ear device of claim 13, wherein the predetermined limit corresponds to a jaw position associated with clenching the jaw or grinding teeth.

18. The custom in-ear device of claim 13, wherein the predetermined limit corresponds to a jaw position associated with the one or more symptoms of the temporomandibular joint disorder.

19. The custom in-ear device of claim 13, wherein the in-ear device is configured to conform to the subject's ear canal with a three-dimensional optical scan of the subject's ear canal when the subject's jaw is in the therapeutic position.

20. The custom in-ear device of claim 13, further comprising at least one sensor that generates a signal when the subject's jaw moves past the predetermined limit.

21. The custom in-ear device of claim 20, wherein the at least one sensor comprises at least one force sensor that senses force on the subject's ear canal associated with the movement of the subject's jaw.

22. The custom in-ear device of claim 20, wherein the at least one sensor comprises at least one accelerometer that detects acceleration of the subject's jaw.

23. The custom in-ear device of claim 20, wherein the at least one sensor is a voltage sensor that senses a voltage across the subject's muscles or nerves.

24. The custom in-ear device of claim 20, wherein the signal is an auditory signal or a vibration.

25. The custom in-ear device of claim 20, wherein the in-ear device comprises a generally C-shaped interior cavity.

26. A method of manufacturing an in-ear device designed to generate proprioceptive cues in the ear of a subject having temporomandibular joint disorder, comprising:
customizing the in-ear device so it conforms to the subject's ear canal when the subject's jaw is in a therapeutic position, wherein the therapeutic position is a position free of one or more symptoms of the temporomandibular joint disorder; and
configuring at least one feature of the in-ear device so that the at least one feature provides a cue to the subject when the subject's jaw moves past a predetermined limit in range of motion.

27. The method of claim 26, wherein the predetermined limit corresponds to a jaw position associated with the one or more symptoms of the temporomandibular joint disorder.

28. The method of claim 26, further comprising providing a protrusion to a surface of the in-ear device at an angle that is selected based on the subject's ear canal shape and dimensions.

29. The method of claim 28, further comprising providing a protrusion to a surface of the in-ear device at an angle that is selected based on the subject's demographics.

30. The method of claim 26, further comprising:
optically scanning the subject's ear canal to generate a three dimensional image based on the scan; and
configuring the in-ear device based on the generated image.

31. The method of claim 30, wherein the subject's jaw is in the therapeutic position during the scanning.

32. The method of claim 30, further comprising inserting a mechanical device into the subject's ear canal to measure parameters of the subject's ear canal and configuring the in-ear device based on the measured parameters.

33. A method of reducing symptoms of temporomandibular joint disorder in a subject comprising:
providing, by the in-ear device, a sensory indication to the subject that the subject's jaw has moved past a predetermined limit in a range of motion.

34. The method of claim 33, further comprising:
determining, by an in-ear device, when the subject's jaw moves past the predetermined limit, and
generating a signal by a sensor of the in-ear device when the subject's jaw moves past the predetermined limit.

35. The method of claim 33, further comprising deforming the subject's ear canal, by the in-ear device, when the subject's jaw moves past the predetermined limit.

36. A method of reducing symptoms of temporomandibular joint disorder in a subject comprising:
providing, by the in-ear device, a sensory indication to the subject at predetermined intervals.

37. The method of claim 36, further comprising generating an auditory signal or vibration or both.

38. A proprioceptive for insertion in a subject's ear canal, wherein the proprioceptive is configured to substantially deform the subject's ear canal when the subject's jaw is not in a therapeutic position, wherein the therapeutic position is between an extreme open position and an extreme closed position of the jaw and wherein the therapeutic position is a position free of one or more symptoms of temporomandibular joint disorder in the subject.
